(19) United States
(12) Patent Application Publication
(10) Pub. No.: US 2010/0292183 A1
(43) Pub. Date: Nov. 18, 2010

(54) TETRACYCLINE AND USES THEREOF

(76) Inventor: Shanmugavel MADASAMY, Cupertino, CA (US)

Correspondence Address: WILSON, SONSINI, GOODRICH & ROSATI, 650 PAGE MILL ROAD, PALO ALTO, CA, 94304-1050, US

(21) Appl. No.: 12/780,733

(22) Filed: May 14, 2010

Related U.S. Application Data
(60) Provisional application No. 61/178,893, filed on May 15, 2009.

(51) Int. Cl. A61K 31/65; C07D 495/04; C07D 401/12; A61K 31/727; C07H 3/06; A61P 9/10; A61P 7/02

(52) U.S. Cl. 514/56; 552/203; 514/152; 552/206; 552/202; 536/21

(57) ABSTRACT

Provided herein are methods and compositions for the in vitro formation of tetracycline conjugates, for example, tetracycline conjugated to anti-coagulants. The present invention also provides methods for preventing or treating plaque-related diseases or disorders such as atherosclerosis using tetracycline alone, or the tetracycline conjugates of the present invention.

### CROSS REFERENCE

This application claims the benefit of U.S. Provisional Patent Application No. 61/178,893, entitled “Tetracycline conjugates and uses thereof” filed May 15, 2009, the contents of which are incorporated by reference in their entirety.

### BACKGROUND OF THE INVENTION

Assembly of pathogenic complexes, broadly described as “plaques”, causes diseases such as atherosclerosis and Alzheimer's disease. Atherosclerosis is a disease of the arteries that results from inflammation and the build-up of plaque under the lining of the blood vessel. The development of atherosclerotic plaques in the walls of blood vessels begins early in childhood and continues to grow asymptomatically through the mid age. These plaques consist of low-density lipoproteins, decaying muscle cells, fibrous tissue, and clumps of blood platelets, cholesterol, and calcified nodules. They tend to form in regions of turbulent blood flow and are found most often in people with high concentrations of cholesterol in the bloodstream. The number and thickness of plaques increase with age, causing loss of the smooth lining of the blood vessels. As patients age, they develop a number of chronic cardiovascular complications due to advancement of the atherosclerotic plaques. Furthermore, when these unstable atherosclerotic plaques rupture, atherothrombus (blood clot) is generated that occludes blood vessels and results in life-threatening conditions such as myocardial infarction, stroke and thromboembolic events.

Atherosclerosis is the leading cause of death in the developed world, and atherosclerosis is predicted to be the leading cause of death in the developing world within the first quarter of this century. According to the American Heart Association, coronary heart disease affects nearly 14 million people in the United States and more than 30 million worldwide. In the United States, approximately 1.5 million myocardial infarctions occur annually. Atherosclerotic interference with blood supply to the brain (stroke) is the third most common cause of death after cancer. Atherosclerosis is responsible for more than half of the yearly mortality in the United States, and more than 500,000 people die annually of myocardial infarction alone. This rate of mortality costs the country more than $100 billion a year. Atherosclerosis is rapidly increasing in prevalence in developing countries, and as people in developed countries live longer, incidence will increase. By 2020, atherosclerosis is expected to be the leading cause of death worldwide.

Atherosclerosis typically does not produce obvious symptoms until the damage to the arteries is severe enough to restrict blood flow. Medications for treating atherosclerosis offer limited help, since the damage has already been done. Currently, patients with atherosclerotic plaques are treated mainly with statins, anti-thrombin and anti-platelet aggregation. Anticoagulant drugs have been used to minimize secondary clotting and embolus formation. The anti-platelet aggregation drugs or blood thinners prevent blood platelet aggregation involved in the blood clot formation. Commonly used blood thinner drugs to treat patients with risk of developing second heart attack or stroke include Clopidogrel (Plavix), Acenocoumorol (Warfarin), Dabigatran Etelixate (Pradax), Heparin (Lovenox), Ticlopidine, Prasugrel (Effient), Asprin and Elinogrel (Phase II clinical study). Although effective, numerous clinical studies have reported that patients taking these drugs are at risk to develop severe bleeding that could cause life threatening conditions (Wodlinger et al. Chu et al. Pickard et al. DeEugenio et al.). There is thus an unmet medical need for new and improved anti-coagulants therapeutics for patients at risk for atherosclerosis and related atherothrombosis indications.

### INCORPORATION BY REFERENCE

All publications, patents, and patent applications mentioned in this specification are herein incorporated by reference in their entirety, to the same extent as if each individual publication, patent, or patent application was specifically and individually indicated to be incorporated by reference.

### SUMMARY OF THE INVENTION

In one aspect, the present invention provides a composition for preventing or treating a plaque-related disease or disorder, the composition comprising tetracycline or a derivative, salt, or analog of tetracycline. In some embodiments, the tetracycline, or derivative, salt, or analog thereof, is conjugated to an anticoagulant. In some embodiments of the composition, the tetracycline salt is tetracycline chloride. In some embodiments, the anticoagulant is selected from the group consisting of clopidogrel, warfarin (coumadin), acenocoumarol, elinogrel, enoxaparin, prasugrel, phenprocoumon, brodifacoum, phenindione, heparin, fondaparinux, idraparinux, argatroban, lepirudin, bivalirudin, dabigatran, dabigatran etexilate, ximelagatran, and any salt, derivative or analog thereof. In some embodiments, the tetracycline or a derivative, salt, or analog of tetracycline is covalently conjugated to an anticoagulant. In some embodiments, the tetracycline or a derivative, salt, or analog of tetracycline is covalently conjugated to an anticoagulant via a linker. The linker can be an acid labile linker, an ester linker, a hydrazone linker, a sulfonamide-containing linker, an enzymatically cleavable linker, or a polymer based linker. In some embodiments, the tetracycline-anticoagulant conjugate binds, penetrates, disassembles, prevents or disrupts a plaque. In some embodiments, the tetracycline-anticoagulant conjugate inhibits thrombosis. In some embodiments, the plaque-related disease or disorder is atherosclerosis or atherothrombosis. The plaque-related disease or disorder can also be Alzheimer's disease, kidney stone, gall bladder stone, pancreatic stone, or a calcification-related disease or disorder. In some embodiments, the composition of the present invention further comprises a pharmaceutically acceptable excipient or carrier. In some embodiments, the tetracycline or a derivative, salt, or analog thereof targets the anticoagulant to sites of plaques. In some embodiments, the tetracycline-anticoagulant conjugate, upon administration to a subject, exhibits reduced side effects and toxicity as compared to administration of unconjugated tetracycline and the anticoagulant individually. In some embodiments, the tetracycline-anticoagulant conjugate, upon administration to a subject, improves pharmacokinetic profile of the anticoagulant as compared to administration of the anticoagulant alone. In some embodiments, the tetracycline-anticoagulant conjugate, upon administration to a subject, increases half-life of the anticoagulant as compared to administration of the anticoagulant alone.

In another aspect, the present invention provides a method of generating a conjugate for preventing or treating a plaque-related disease or disorder, the method comprising covalently linking tetracycline or a derivative, salt, or analog of tetracycline to an anticoagulant. In another aspect, the present invention provides a method of preventing or treating a plaque-related disease or disorder comprising administering to a subject an effective amount of a tetracycline or a derivative, salt, or analog thereof. In some embodiments, the tetracycline, or derivative, salt or analogy thereof, is conjugated to an anticoagulant.

In practicing any of the subject methods of the present invention, in some embodiments, the tetracycline salt is tetracycline chloride. In some embodiments, the anticoagulant is selected from the group consisting of clopidogrel, warfarin (coumadin), acenocoumarol, elinogrel, enoxaparin, prasugrel, phenprocoumon, brodifacoum, phenindione, heparin, fondaparinux, idraparinux, argatroban, lepirudin, bivalirudin, dabigatran, dabigatran etexilate, ximelagatran, and any salt, derivative or analog thereof. In some embodiments, the tetracycline or a derivative, salt, or analog thereof is covalently conjugated to an anticoagulant via a linker. The linker can be an acid labile linker, an ester linker, a hydrazone linker, a sulfonamide-containing linker, an enzymatically cleavable linker, or a polymer based linker. In some embodiments, the tetracycline-anticoagulant conjugate binds, penetrates, disassembles, prevents or disrupts a plaque. In some embodiments, the tetracycline-anticoagulant conjugate inhibits thrombosis. In some embodiments, the plaque-related disease or disorder is atherosclerosis or atherothrombosis. The plaque-related disease or disorder can also be Alzheimer's disease, kidney stone, gall bladder stone, pancreatic stone, or a calcification-related disease. In some embodiments, the tetracycline or a derivative, salt, or analog thereof, upon conjugation to an anticoagulant, targets the anticoagulant to sites of plaques. In some embodiments, the tetracycline-anticoagulant conjugate, upon administration to a subject, exhibits reduced side effects, toxicity or bleeding as compared to administration of unconjugated tetracycline and the anticoagulant individually. In some embodiments, the tetracycline-anticoagulant conjugate, upon administration to a subject, increases half-life of the anticoagulant as compared to administration of the anticoagulant alone. In some embodiments, the subject is a mammal. In some embodiments, the subject is a human. In some embodiments, the administration of the conjugate is oral or parenteral injection.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic drawing showing the tetracycline-anti-coagulant conjugate via a covalent linker. The resultant conjugate binds to atherosclerotic plaques and inhibits thrombosis. This schematic depicts one embodiment of the present disclosure.

### DETAILED DESCRIPTION OF THE INVENTION

In one aspect, the present invention provides a composition for preventing or treating a plaque-related disease or disorder comprising tetracycline or a derivative, salt, or analog of tetracycline conjugated to an anticoagulant. In another aspect, the present invention provides a method of generating a conjugate for preventing or treating a plaque-related disease, the method comprising covalently linking tetracycline or a derivative, salt, or analog of tetracycline to an anticoagulant. In another aspect, the present invention provides a method of preventing or treating a plaque-related disease comprising administering to a subject an effective amount of a conjugate or a pharmaceutical composition thereof, wherein the conjugate comprises tetracycline or a derivative, salt, or analog of tetracycline conjugated to an anticoagulant. In still another aspect, the present invention provides a method of preventing or treating a plaque-related disease comprising administering an effective amount of tetracycline or a derivative, salt, or analog thereof, to a subject afflicted with, suspected of being afflicted with, or at risk for a plaque-related disease or disorder.

### I. Plaque-Related Diseases or Disorders

The present invention provides a composition and a method of treating a plaque related disease or disorder. Numerous types of diseases or disorders are associated with plaque formation, as listed in Table 1.

TABLE 1

| Amyloid aggregates | Chemical aggregates | Lipid aggregates | Protein aggregates |
|---|---|---|---|
| Abeta40-42 | Calcium phosphate | Phospholipids | Serum Amyloid A |
| (Alzheimer's) | (Atherosclerosis) | (Atherosclerosis) | (Systemic Amyloidosis) |
| Synuclein | Calcium oxalate | Cholesterol crystals | Beta Microglobulin |
| (Parkinson's)) | (Kidney Stones) | (Atherosclerosis, Gall | (Dialysis related |
| | | Bladder) | Amyloidosis) |
| Prion | Calcium Pyrophosphate | Palmitic anhydrate | Lysozyme |
| (Mad Cow Spongiform) | (Osteoarthritis) | (Gall bladder stone) | (Lysozyme Amyloidosis) |
| Amylin | Calcium Bicarbonate | | Insulin |
| (Type II diabetes) | (Pancreatic stones) | | (Insulin related |
| | | | Amyloidosis) |
| Tau | Ammonium Phosphate | | Super dioxide dismutase |
| (Dementia/parkinsonism) | (Kidney stone disease) | | (Amyotrophic Lateral |
| | | | sclerosis) |

### Atherosclerosis

In some embodiments, the composition and methods of the present invention can be used for preventing or treating atherosclerosis. In some embodiments, the tetracycline-anticoagulant of the present invention can be used to prevent the formation of an atherosclerotic plaque related thrombosis.

Atherosclerosis is a disease affecting arterial blood vessels. It is commonly referred to as a hardening of the arteries. It is caused by the formation of multiple plaques within the arteries. As used herein, the term “atherosclerotic plaque” refers to the build up of fatty plaque deposits within the wall of blood vessels. The major components present in atherosclerotic plaques are lipids, cholesterol, calcium crystals, proteins, blood platelets, fibrin, blood clots, macrophages, cell debris, microorganisms and minerals (Corti R et al, 2004). Although, the chemical and physical nature of a plaque's contents varies greatly, a common factor responsible for origin of the plaque diseases or disorders is formation of insoluble aggregates or materials. Examples of such insoluble aggregates that are implicated in the origin of many human diseases or disorders are listed in table 1. Insoluble aggregate formation takes place, under abnormal or pathological conditions, either as self aggregation of the homogeneous molecules or as by-products derived from interaction between two or more molecules. For example, cholesterol and calcium phosphate (CP) crystals represent two major components accumulated in the advanced atherosclerotic plaques and the accumulation of former is due to its self aggregation whereas the latter is a by-product resulted from interaction between inorganic calcium and phosphate. Numerous studies have reported the important role of cholesterol and CP either individually or in combination towards the development of atherosclerotic processes (Doherty, T M et al, 2004).

Plasma lipids and lipoproteins are associated with an increased risk of cardiovascular diseases or disorders. These lipid molecules exist both as low-density lipoprotein (LDL) particles and elevated plasma triglyceride. Further, studies have found heterogeneities in size, density and composition among LDL particles (Mkkanen L et al, 1999) and particularly the small LDL particles are found to be more atherogenic than the larger LDL particles (Carmena R et al, 2004). Cholesterol is a major lipid component present in the atherosclerotic plaques and specifically, the low-density cholesterol particles are found to be associated with increased risk of coronary heart diseases (Hirano T et al, 2003; Festa A et al, 1998). The formation of a lipid-rich lesion in the artery wall is considered to be a key event in the initiation and progression of atherosclerosis (Peng S et al, 2000). Continuous accumulation of lipids and cholesterol in an atherosclerotic plaque may cause progressive narrowing of the arterial lumen. It has been hypothesized that during the atherosclerotic process, cholesterol undergoes a structural transition from the initial esterified form to an intermediate un-esterified aggregates form and finally to an irreversible crystal form (Sarig S et al, 1994a). The plaque-containing major share of un-esterified cholesterol aggregates in the lipid core is considered vulnerable for rupture and thrombus formation (Small D M, et al, 1988).

In addition, the composition of the atherosclerotic plaques is an important factor in predicting the stability of the plaques. Apart from the presence of heterogenic chemical constituents in the atherosclerotic plaques, the physical nature of each component also exists in various forms. For example, the cholesterol is present in the atherosclerotic plaques as noncrystalline and crystalline monohydrate forms and the former is largely localized in the soft plaque core making it susceptible for rupture (Guyton, J R, 1996). Inside the atherosclerotic plaques core, the cholesterol particles are found to occur in three different sizes such as small spherulites (3-5 μm), elongated structures (10-30 μm) and large irregular deposits (100 μm) (Sarig S, et al, 1994b). Further, agglomeration of these un-esterified cholesterol particles along with granular CP is a common indication found in the advanced plaques (Kruth H S, 1984). However, the physical association between cholesterol and CP in the atherosclerotic plaques development need to be further elucidated (Hirsch D, 1993). Advanced analytical tools such as X-ray diffraction, optical microscopy and Raman spectroscopy are useful to identify and semi-quantitate the crystalline contents of cholesterol and CP in the matured atherosclerotic plaques (Guo W et al, 2000).

Atherosclerosis is a major cardiovascular complication and a leading cause of death among patients with the chronic kidney disease and end stage renal disease (ESRD) thus revealing casual association between kidney and heart diseases (Brancaccio D et al, 2005). Increasing evidence has shown that due to abnormal minerals metabolism, the serum concentrations of calcium and phosphate are present in elevated levels in patients with the chronic kidney disease and hemodialysis (Young E W et al, 2005). The formation of disordered extra skeletal calcium crystals or stones is a common medical problem causing various chronic diseases or disorders such as atherosclerosis, kidney and bladder stones, dental pulp stones, some gall stones, salivary gland stones, chronic calculus prostatitis, scleroderma, pancreatic stones, several malignancies, pseudogout etc (Carson D A, 1998). The CP crystals are present in the approximately 82% of the advanced atherosclerotic plaques and remain co-localized with fibrin and cholesterol deposits (Bini A et al, 1999). These crystals deposited in the entire region of vascular system found to cause chronic cardiovascular pathologies such as stiffening of the arteries, stenosis, reduced blood flow, altered coronary perfusion and under acute conditions causes heart attack and stroke (Higgins C L et al, 2005). According to the American Heart Association, the coronary calcification and particularly aortic valve calcification is the third leading cause of the heart diseases or disorders in adults (Garg V et al 2005). Further estimates show that approximately 40-50% of mortalities among patients with chronic kidney disease are due to cardiovascular complications such as atherosclerosis, valvular calcification, myocardial infarction etc (Campean V et al, 2005). Particularly, elderly patients with the chronic kidney disease and ESRD are vulnerable for the development of vascular calcification and atherosclerosis revealing the link between kidney and heart diseases (Cullen P et al, 2005). Abnormal metabolisms of both lipids and minerals play significant roles in the atherosclerotic processes. The accumulation of lipid, cholesterol and calcium-containing crystals in the atherosclerotic plaques and the clinical consequences of their exposure to the hemostasis factors during plaques rupture are life threatening. In addition to abnormal lipid and mineral metabolisms, inappropriate activation of blood coagulation cascade, vascular injury, thrombosis and endothelial dysfunctions attributed to early events of atherosclerotic plaque formation (Xiao Q et al, 1998). Dyslipidemia plays important roles in the pathogenesis of atherosclerosis and specifically, the low-density cholesterol particles are found to be associated with increased risk of coronary heart diseases (Festa A et al, 1998). Improved targeting to atherosclerotic plaques and effective inhibition of thrombosis are needed to develop novel therapeutics to treat atherosclerosis and its related cardiovascular diseases or disorders.

### Thrombosis

In some embodiments, the present invention provides compositions and methods for inhibiting thrombosis. In one embodiment, the tetracycline-anticoagulant conjugate binds, penetrates, disassembles, prevents or disrupts a plaque. In another embodiment, the tetracycline-anticoagulant conjugate inhibits thrombosis.

Thrombosis, i.e. blood clot formation, is a natural defensive mechanism in the body to prevent excessive bleeding due to injury or infections. Atherothrombosis is a serious medical condition occurring due to the rupture of atherosclerotic plaques contributing to the development of heart attack and stroke. Antiplatelet agents like aspirin and clopidogrel are treatment cornerstones for acute coronary syndromes and widely used in secondary prevention of ischemic events. Anti-coagulants are commonly prescribed to patients diagnosed with atherosclerotic plaque related cardiovascular complications such as unstable angina, stent fixed, and first heart attack or stroke symptoms. Although the objective of using anti-coagulants is to prevent the formation of intra vascular blood clot due to unstable plaque rupture, the medication subjects the entire blood coagulation system of the body to a prolonged state of suppression. This non-selective inhibition of blood clot formation exhibited by currently available anti-coagulant drugs is the main cause of drug induced systemic hemorrhage. Therefore, there exists a need to develop safe, selective and targeted anti-coagulation drugs that would significantly reduce the risk of developing systemic bleeding in patients during the course of medication.

In some embodiments, tetracycline-anticoagulant conjugate of the present invention can selectively target and bind to atherosclerotic plaques and the anticoagulant can inhibit thrombosis.

### II. Tetracycline Conjugates

In one aspect, the present invention provides a composition for preventing or treating a plaque-related disease or disorder including atherosclerosis, the composition comprising tetracycline or a derivative, salt, or analog of tetracycline conjugated to an anticoagulant.

In some embodiments, the conjugate agent of the present invention can be used to prevent or treat atherosclerosis and atherothrombosis by inhibiting platelet aggregation. The strategy for developing safe and improved anti-platelet aggregation drug is based on the understanding that targeting the anti-coagulants to the site/s of atherosclerotic plaque in the arteries would minimize the severe side effects of these drugs. Tetracycline is a broad-spectrum antibiotic mostly used for treating bacterial infections. In vitro and in vivo plaque studies have shown that tetracycline has specific affinity binding to advanced atherosclerotic plaques (disclosed in U.S. patent application Ser. No. 12/286,368, which is herein incorporated by reference in its entirety).

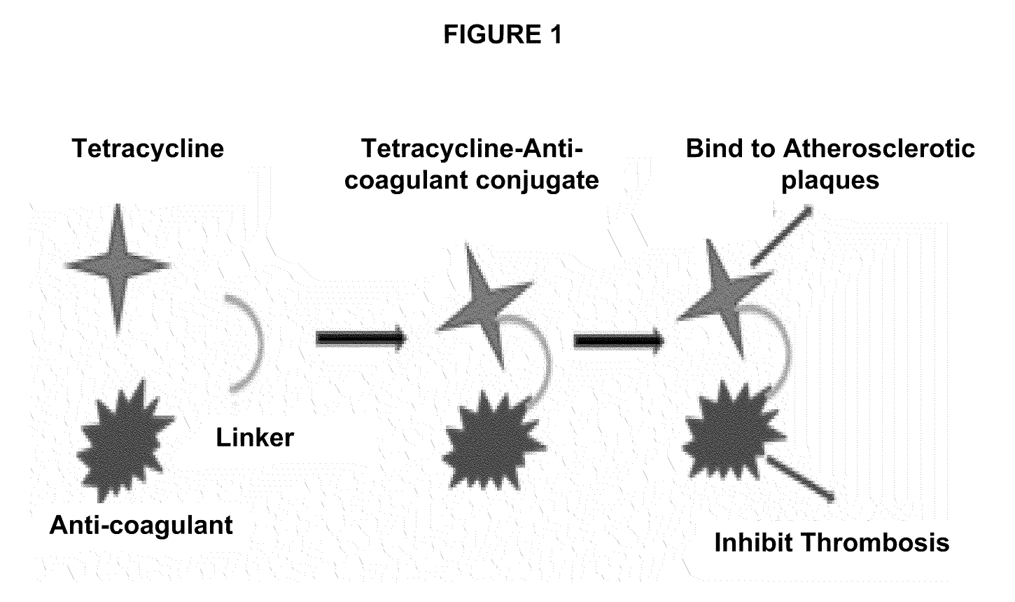

The present invention generally relates to generating improved anti-coagulant drugs involving covalent coupling of tetracycline or its non-antibacterial derivatives with anti-coagulation factors including but not limited to anti-platelet aggregation drugs, anti-thrombin drugs, and inhibitors of blood coagulation proteins to selectively target these drugs to the site/s of advanced atherosclerotic plaques. The resultant tetracycline-anticoagulant conjugates have dual functions (FIG. 1). First, the tetracycline moiety of the conjugate selectively targets the conjugate binding to the advanced atherosclerotic plaques. Second, the anti-coagulant moiety inhibits blood coagulation thus preventing atherothrombosis if the plaque ruptures.

The tetracycline or tetracycline derivative or analog conjugate of the present invention has several advantages. In some embodiments, the tetracycline conjugate is a tetracycline-anti-coagulant conjugate. In some embodiments, the tetracycline conjugate of the present invention selectively targets a drug to the sites of advanced atherosclerotic plaques in the arteries to prevent the risk of developing atherothrombosis. Second, inhibition of atherothrombosis is limited to the site of atherosclerotic plaques during plaque rupture leaving the blood coagulation system largely unaffected. Third, the conjugates of the present invention are used at lower doses as compared to their parent molecules used individually, which reduces the systemic bleeding in the body thus resulting in improved safety and toxicity profile. Fourth, binding of the conjugates to the plaques increases the half-life of the drugs, for example, the half-life of an anticoagulant, thereby providing patients a longer period of protection against the risk of developing atherothrombosis. In addition, the tetracycline-anticoagulant conjugates provide clinical advantages as compared to both currently available anti-coagulants and the newer agents that are in clinical development, such as Prasugrel, by lowering the risk of ischemic events due to clot formation and reducing the risk of bleeding. In some embodiments, the tetracycline-anticoagulant conjugate, upon administration to a subject, exhibits reduced side effects and toxicity as compared to administration of unconjugated tetracycline and the anticoagulant individually. In some embodiments, the tetracycline-anticoagulant conjugate, upon administration to a subject, improves pharmacokinetic profile of the anticoagulant as compared to administration of the anticoagulant alone.

### Tetracycline

Tetracycline is a broad-spectrum polyketide antibiotic produced by the Streptomyces genus of Actinobacteria, indicated for use against many bacterial infections. It is sold under the brand names Sumycin, Terramycin, Tetracyn, and Panmycin, among others. Actisite is a thread-like fiber form, used in dental applications. It is also used to produce several semi-synthetic derivatives, which together are known as the tetracycline antibiotics. Tetracycline works by inhibiting action of the prokaryotic 30s ribosome, by binding the 16S rRNA thereby blocking the aminoacyl-tRNA. However, bacterial strains can acquire resistance against tetracycline and its derivates by encoding a resistance operon. In eukaryotic cells, toxicity may be the result of inactivation of mitochondrial 30S ribosomes.

There are many side effects associated with tetracycline antibiotics group. The side effects include but are not limited to stain on developing teeth, inactivation by Ca²⁺ ion so that it can not be taken with milk or yogurt; inactivated by aluminium, iron and zinc, not to be taken at the same time as indigestion remedies; inactivation by common antacids and over-the-counter heartburn medicines; skin photosensitivity; drug-induced lupus and hepatitis; tinnitus; dry and flaky skin; interference with methotrexate by displacing it from the various protein binding sites; breathing complications as well as anaphylactic shock in some individuals; and effect on bone growth of fetus.

In genetic engineering tetracycline is used in transcriptional activation. Tetracycline is also one of the antibiotics used to treat ulcers caused by bacterial infections. In cancer research, tetracycline has been used to reliably cause regression of advanced stages of leukemia in mice, by putting this inexpensive antibiotic into their drinking water.

Tetracycline has been identified for the treatment of atherosclerosis. Its anti-atherosclerotic property in the biochemical, cell culture and mice models of atherosclerosis has been disclosed in US Patent Application Publication No. US2009-0104121-A1, which is herein incorporated by reference in its entirety.

The tetracycline derivatives that can be used in the present invention include but are not limited to 6-demethyltetracycline, bromotetracycline, chlorotetracycline, clomocycline, demeclocycline, doxycycline, lymecycline, meclocycline, methacycline, minocycline, oxytetracycline, rolitetracycline, tetracycline, sancycline, 5a,6-anhydrotetracycline, DDA-tetracycline, dactylocyclinone and 8-methoxychlorotetracycline. The tetracycline derivative also includes the optical isomers of the compounds mentioned above. More preferably, the tetracycline derivative is selected from doxycycline, methacycline, sancycline and minocycline. Even more preferably, the tetracycline derivative is selected from 7-halosancycline, 9-halosancycline and minocycline. Most preferably, the tetracycline derivative is minocycline or any tetracycline derivative or analog or tetracycline conjugated to anti-coagulants (e.g, warfarin, acenocoumarol, clopidogrel).

### Anti-Coagulants

The present invention generally relates to a composition and methods for preventing or treating a plaque-related disease or disorder comprising tetracycline or a derivative, salt, or analog of tetracycline conjugated to an anticoagulant, as well as to methods for preventing or treating a plaque-related disease or disorder by administering to a subject tetracycline or a derivative, salt, or analog thereof.

An anticoagulant is a substance that prevents coagulation; that is, it stops blood from clotting. A group of pharmaceuticals called anticoagulants can be used in vivo as a medication for thrombotic disorders. Anticoagulants are given to people to stop thrombosis (blood clotting inappropriately in the blood vessels). This is useful in primary and secondary prevention of deep vein thrombosis, pulmonary embolism, myocardial infarctions and strokes in those who are predisposed.

There are several classes of anticoagulants. One class includes coumarines (Vitamin K antagonists). The oral anticoagulants are a class of pharmaceuticals that act by antagonizing the effects of vitamin K. Examples include warfarin. It is important to note that it takes at least 48 to 72 hours for the anticoagulant effect to develop fully. In cases when any immediate effect is required, heparin must be given concomitantly. Generally, these anticoagulants are used to treat patients with deep-vein thrombosis (DVT), pulmonary embolism (PE), atrial fibrillation (AF), and mechanical prosthetic heart valves. Warfarin (Coumadin) is the main agent used in the U.S. and UK. Other anticoagulants include but are not limited to acenocoumarol, phenprocoumon, brodifacoum and phenindione. There are adverse effects associated with these anticoagulants. Patients aged 80 years or more may be especially susceptible to bleeding complications with a rate of 13 bleeds per 100 person-years.

Another class of anticoagulants includes heparin and derivative substances. Heparin is a biological substance, usually made from pig intestines. It works by activating antithrombin III, which blocks thrombin from clotting blood. Heparin can be used in vivo and also in vitro to prevent blood or plasma clotting in or on medical devices. Vacutainer brand test tubes containing heparin are usually colored green. Low molecular weight heparin is a more highly processed product that is useful as it does not require monitoring of the APTT coagulation parameter (it has more predictable plasma levels) and has fewer side effects. There are also synthetic pentasaccharide inhibitors of factor Xa. For example, fondaparinux is a synthetic sugar composed of the five sugars (pentasaccharide) in heparin that bind to antithrombin. It is a smaller molecule than low molecular weight heparin.

Another type of anticoagulant is the direct thrombin inhibitor. Current members of this class include but are not limited to argatroban, lepirudin, bivalirudin, and dabigatran.

In one example, the tetracycline or a derivative or analog of tetracycline is conjugated to clopidogrel. Clopidogrel is an oral antiplatelet agent (thienopyridine class) to inhibit blood clots in coronary artery disease, peripheral vascular disease, and cerebrovascular disease. Clopidogrel is a pro-drug whose action may be related to adenosine diphosphate (ADP) receptor on platelet cell membranes. The specific subtype of ADP receptor that clopidogrel irreversibly inhibits is P2Y₁₂ and is important in platelet aggregation and the cross-linking of platelets by fibrin (Savi, P; et al. 2006). The blockade of this receptor inhibits platelet aggregation by blocking activation of the glycoprotein IIb/IIIa pathway. The IIb/IIIa complex functions as a receptor mainly for fibrinogen and vitronectin but also for fibronectin and von Willebrand factor. Activation of this receptor complex is the “final common pathway” for platelet aggregation, and is important in platelet aggregation, the cross-linking of platelets by fibrin. Platelet inhibition can be demonstrated two hours after a single dose of oral clopidogrel, but the onset of action is slow, so that a loading-dose of 300-600 mg is usually administered. Clopidogrel is indicated for prevention of vascular ischaemic events in patients with symptomatic atherosclerosis, acute coronary syndrome without ST-segment elevation (NSTEMI), along with aspirin, and ST elevation MI (STEMI). It is also used, along with aspirin, for the prevention of thrombosis after placement of intracoronary stent. Serious adverse drug reactions associated with clopidogrel therapy include severe neutropenia, Thrombotic thrombocytopenic purpura (TTP) and hemorrhage—gastrointestinal hemorrhage and cerebral hemorrhage.

In another example, the tetracycline or a derivative or analog of tetracycline is conjugated to dabigatran. Dabigatran is an anticoagulant from the class of the direct thrombin inhibitors. It is being studied for various clinical indications, for some of which it may replace warfarin as the preferred anticoagulant. It is orally administered as the prodrug dabigatran etexilate. Dabigatran was discovered from a panel of chemicals with similar structure to benzamidine-based thrombin inhibitor α-NAPAP (N-alpha-(2-naphthylsulfonylglycyl)-4-amidinophenylalanine piperidide. Study showed a good safety profile at doses between 12.5 and 300 mg of dabigatran twice daily. The National Health Service in Britain has authorised the use of dabigatran for use in preventing blood clots in hip and knee surgery patients.

In another example, the tetracycline or a derivative or analog of tetracycline is conjugated to enoxaparin sodium. Enoxaparin is a low molecular weight heparin marketed as Lovenox or Clexane. It is used to prevent and treat deep vein thrombosis or pulmonary embolism, and is given as a subcutaneous injection. Its use is evolving in acute coronary syndromes (ACS). Enoxaparin binds to and accelerates the activity of antithrombin III. By activating antithrombin III, enoxaparin preferentially potentiates the inhibition of coagulation factors Xa and IIa. The anticoagulant effect of enoxaparin can be directly correlated to its ability to inhibit factor Xa. Factor Xa catalyzes the conversion of prothrombin to thrombin, so enoxaparin's inhibition of this process results in decreased thrombin and ultimately the prevention of fibrin clot formation. There are side effects associated with enoxaparin sodium including but not limited to bleeding, thrombocytopenia, pain, bruising or irritation; and hard, inflamed nodules or an itchy red rash at the injection site.

In another example, the tetracycline or a derivative or analog of tetracycline is conjugated to prasugrel. Prasugrel is a novel platelet inhibitor for acute coronary syndromes planned for percutaneous coronary intervention (PCI). Prasugrel is a member of the thienopyridine class of ADP receptor inhibitors, similar to ticlopidine (trade name Ticlid) and clopidogrel (trade name Plavix). These agents reduce the aggregation of platelets by irreversibly binding to P2Y₁₂ receptors.

In another example, the tetracycline or a derivative or analog of tetracycline is conjugated to acenocoumorol. Acenocoumarol is an anticoagulant that functions as a vitamin K antagonist as described hereinabove. It is a derivative of coumarin and is marketed under the brand names Sintrom and Sinthrome.

### Chemical Conjugation and Linkers

In one aspect, the present invention provides a composition for preventing or treating a plaque-related disease or disorder comprising tetracycline or a derivative, salt, or analog of tetracycline conjugated to an anticoagulant. The term “tetracycline conjugates” as used herein encompasses any tetracycline derivative or analog conjugated to a drug molecule, for example, an anticoagulant. The term “tetracycline-anticoagulant conjugates” as used herein encompasses any tetracycline derivative or analog conjugated to an anticoagulant. Tetracycline or an analog or derivative thereof is used as a carrier molecule to target a drug, for example, an anti-coagulant conjugated to tetracycline via an appropriate linker, to the site of advanced atherosclerotic plaques. Conjugates of small molecule ligand, such as an anticoagulant, have advantages over large molecule conjugates such as antibodies because of their better cell/tissue penetration, serum stability and that small molecules do not typically elicit strong immune response from patients.

The design of a good small molecule conjugate involves several essential elements such as selecting appropriate ligand, drug and linker. In some embodiments, tetracycline is used to target a drug to a plaque, for example, an atherosclerotic plaque. In some embodiments, tetracycline is covalently conjugated to an anticoagulant. In some embodiments, tetracycline is covalently conjugated to an anticoagulant via a linker. Various linkers can be used to couple tetracycline or its derivative or analog to an anti-coagulant covalently. Selection of appropriate linker is essential for successful drug delivery and release. Selection of suitable linkers based on their degradation by endosomes and lysosome pathways has been described in literature (Garnett M C et al; Eaton M et al; Kratz F et al; and Toth I et al.). In some embodiments of the present invention, tetracycline or its derivatives or analogs are used as a scaffold to be coupled with various drugs, for example, anti-coagulant molecules. The chemistry to covalently link various types of anti-coagulants with tetracycline or derivatives or analogs is different for each conjugation synthesis because of the variation in the molecular architecture of the drug molecules. It requires careful selection of both the sites in the tetracycline and anti-coagulants structures and also the choice of linkers for efficient coupling. In some embodiments, the linkers are attached at various sites in the tetracycline or its derivative or analog and the anti-coagulant structures in order to identify tetracycline conjugates with desired properties. After the synthesis, these conjugates are screened in the plaque binding and anti-coagulation assays described in Examples 6-8 for identification of the conjugates that possess the most desirable properties for further drug development.

### Examples of Linkers Used for Synthesis of Tetracycline-Anticoagulant Conjugates

Various linkers can be used to conjugate tetracycline molecule or its derivative or analog to an anti-coagulant molecule. Examples of linkers that can be used in the present invention include but are not limited to acid labile linkers, ester linkers, hydrazone linkers, sulfonamide-containing linkers, enzymatically cleavable linkers, or polymer based linkers.

Acid labile linkers are generally selected for coupling drugs that require biotransformation at acidic environments (pH 4.0-6.0) inside lysozymes (Lavie E et al.). Cis-Aconity linkage was one of the acid labile linker used to make drug conjugates and details of its synthesis, coupling, reagents and methods have been described in the literature (Shen W C et al; Hudecz F et al; Blattler et al; and Srinivasachar K et al). Ester-type linkers are hydrolytically sensitive and have been used to make conjugates with camptothecin, Gembitabine, Doxorubicin and Paclitaxol drugs (Warnecke A et al; Dubowchik G M et al, and Chari R V et al). The other type of acid sensitive linker includes hydrazone linkers. The hydrazone linkers have been used to make conjugates of doxorubicin, morpholinodoxorubicin, streptomycin, 5-fluorouridine, chlorambucil, daunorubicin and vinblastine. The chemistry and reagents used for coupling hydrazone linkers with a drug have been reviewed in Garnett M C et al; Kratz F et al; Greewald et al; Guo P et al and Guo Z et al. Examples of hydrazone linkers include but are not limited to SANH (Succinimidyl 6-hydrazinonicotinamide acetone hydrazone, Formula I), a heterobifunctional crosslinker used to modify amine-containing biomolecules or surfaces to directly incorporate 2-hydrazinopyridine moieties; and SFB (Succinimidyl 4-formylbenzoate, Formula II), a heterobifunctional crosslinker used to modify amine-containing biomolecules or surfaces to directly incorporate benzaldehyde moieties.

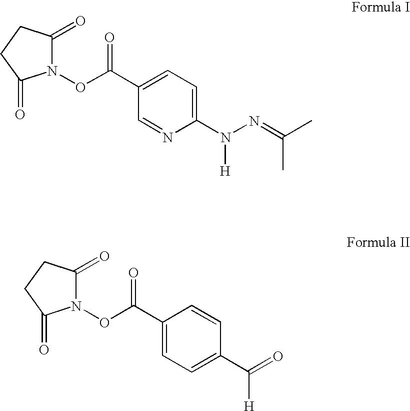

In addition, sulfonamide-containing linkers (Formulae III-V) have been used in drug design and conjugate synthesis with biomolecules (Sum F et al). This class of linkers is available for coupling molecules that require conformational flexibility or rigid conformation.

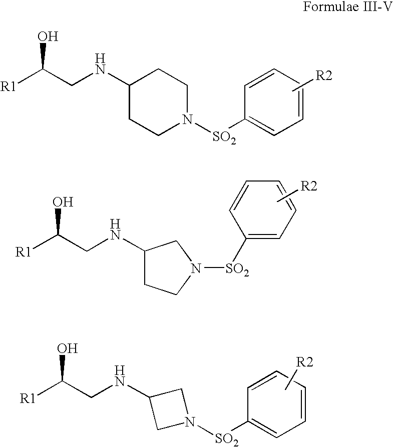

The next class of linkers includes linkers that are enzymatically cleavable by lysosomal proteases such as cathepsin B or D. These proteases cleave specific amino acid sequences introduced into the linkers (Scarborough P E et al). Hydrolase dependent self cleavable linkers and sulphydrl linkers are also used for drug design as reported earlier (Papot et al. and Saito G et al).

In some embodiments of the present invention, polymer-based linkers are used to conjugate tetracycline or its derivative or analog to an anticoagulant molecule. In one example, peglylation i.e. polyethylene-glycol is used to attach tetracycline or its derivative or analog to an anticoagulant molecule. Polymer-based linkers, such as polyethylene-glycol (PEG, Formula VI), are widely used to conjugate both small molecule and large molecule drugs. The PEG conjugated drugs offer a number of desirable advantages including higher solubility, less immunogenicity, improved half-life, targeted delivery and enhanced activity of the drugs. For example, PEGlylation method has been successfully used to conjugate two poorly soluble anti-cancer molecules, paclitaxel and camptothecin, attached to antibody for targeted delivery (Sawant R R et al).

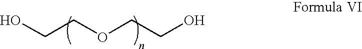

Since the antibiotic activity of tetracycline is not essential to the subject conjugate of the present invention, any side group on the tetracycline molecule can be linked to an anticoagulant. In one example, tetracycline is linked to an anticoagulant via an OH group on tetracycline. In another example, tetracycline is linked to an anticoagulant via the O═C—NH₂ amide group. In another example, tetracycline is linked to an anticoagulant via the amine group on tetracycline. The places on anticoagulant molecules to which tetracycline or its derivative or analog can be linked vary for different anticoagulants. Generally, tetracycline or its derivative or analog is not linked to a biologically active group that is responsible for the anticoagulant activity of the anticoagulant molecule. For example, the O═C—O—CH₃ group on clopidogrel molecule is the biologically active group that is responsible for the anticoagulant activity of clopidogrel. Therefore tetracycline or its derivative or analog can be linked to a group other than O═C—O—CH₃ on clopidogrel such that the anticoagulant activity of clopidogrel will not be affected. In one embodiment, tetracycline is linked via the chloro (Cl) group on clopidogrel to form the tetracycline-clopidogrel conjugate of the present invention.

### Pharmaceutical Composition

Compounds of the invention may be administered as pharmaceutical formulations including those suitable for oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999).

In various embodiments, the pharmaceutical composition includes carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In some embodiments, the pharmaceutical preparation is substantially free of preservatives. In other embodiments, the pharmaceutical preparation may contain at least one preservative. General methodology on pharmaceutical dosage forms is found in Ansel et al., Pharmaceutical Dosage Forms and Drug Delivery Systems (Lippencott Williams & Wilkins, Baltimore Md. (1999)). It will be recognized that, while any suitable carrier known to those of ordinary skill in the art may be employed to administer the compositions of this invention, the type of carrier will vary depending on the mode of administration.

Compounds may also be encapsulated within liposomes using well-known technology. Biodegradable microspheres may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252.

The compound may be administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. Essentially, the material is dissolved in an aqueous solution, the appropriate phospholipids and lipids added, along with surfactants if required, and the material dialyzed or sonicated, as necessary. A review of known methods is provided by G. Gregoriadis, Chapter 14, “Liposomes,” Drug Carriers in Biology and Medicine, pp. 2.sup.87-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

The concentration of drug may be adjusted, the pH of the solution buffered and the isotonicity adjusted to be compatible with intravenous injection, as is well known in the art.

The compounds of the invention may be formulated as a sterile solution or suspension, in suitable vehicles, well known in the art. The pharmaceutical compositions may be sterilized by conventional, well-known sterilization techniques, or may be sterile filtered. The resulting aqueous solutions may be packaged for use as is, or lyophilized, the lyophilized preparation being combined with a sterile solution prior to administration. Suitable formulations and additional carriers are described in Remington “The Science and Practice of Pharmacy” (20^{th} Ed., Lippincott Williams & Wilkins, Baltimore Md.), the teachings of which are incorporated by reference in their entirety herein.

The agents or their pharmaceutically acceptable salts may be provided alone or in combination with one or more other agents or with one or more other forms. For example a formulation may comprise one or more agents in particular proportions, depending on the relative potencies of each agent and the intended indication. For example, in compositions for targeting two different host targets and where potencies are similar, about a 1:1 ratio of agents may be used. The two forms may be formulated together, in the same dosage unit e.g., in one cream, suppository, tablet, capsule, aerosol spray, or packet of powder to be dissolved in a beverage; or each form may be formulated in a separate unit, e.g., two creams, two suppositories, two tablets, two capsules, a tablet and a liquid for dissolving the tablet, two aerosol sprays, or a packet of powder and a liquid for dissolving the powder, etc.

The term “pharmaceutically acceptable salt” means those salts which retain the biological effectiveness and properties of the agents used in the present invention, and which are not biologically or otherwise undesirable. For example, a pharmaceutically acceptable salt does not interfere with the effect of an agent of the invention in preventing, reducing, or destabilizing the formation of a multi-subunit complex, or promoting the disruption of a multi-subunit complex.

Typical salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the agent(s) contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

A pharmaceutically acceptable ester or amide refers to those which retain biological effectiveness and properties of the agents used in the present invention, and which are not biologically or otherwise undesirable. For example, the ester or amide does not interfere with the beneficial effect of an agent of the invention in preventing, reducing or destabilizing assembly of the multi-subunit complex, or promoting disruption or elimination of the multi-subunit complex in the cells, or preventing or alleviating one or more signs or pathological symptoms associated with exposure to one or more multi-subunit complexes or insoluble components in a subject. Typical esters include ethyl, methyl, isobutyl, ethylene glycol, and the like. Typical amides include unsubstituted amides, alkyl amides, dialkyl amides, and the like.

### Kit

The present invention also embodies a kit comprising the conjugate of the present invention for prevention or treatment of a plaque-related disease, disorder, or disease-like process. In some embodiments, the kit comprises one or more tetracycline-anticoagulant conjugates as described herein. In other embodiments, the kit includes a pharmaceutical composition of one or more tetracycline-anticoagulant conjugates with a pharmaceutically acceptable carrier, e.g. salt, adjuvant, booster and the like. In some embodiments, the kit further includes instruments, for example, needles and syringes for in vivo administration of the tetracycline-anticoagulant conjugates. In other embodiments, the present invention provides a kit comprising reagents for chemically synthesizing a tetracycline-drug conjugate, for example, a tetracycline-anticoagulant conjugate. In some embodiments, the reagents include tetracycline or a derivative or analog thereof, an anticoagulant such as clopidogrel, a linker, and buffers or catalysts necessary to carry out the chemical conjugation. In some embodiments, instructions teaching the use of the kit according to the methods described herein are provided. Such kits may also include information, such as scientific literature references, package insert materials, clinical trial results, and/or summaries of these and the like, which indicate or establish the activities and/or advantages of the conjugates of the present invention. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like.

### III. Methods of Use

### A. Method of Generating Tetracycline Conjugates

In some embodiments, the present invention provides a method of generating a conjugate agent for preventing or treating a plaque-related disease or disorder, the method comprising covalently linking tetracycline or a derivative, salt, or analog of tetracycline to an anticoagulant. In one embodiment, the tetracycline salt is tetracycline chloride. The anticoagulant used in the synthesis can be selected from the group consisting of clopidogrel, warfarin (coumadin), acenocoumarol, enoxaparin, prasugrel, phenprocoumon, brodifacoum, phenindione, heparin, fondaparinux, idraparinux, argatroban, lepirudin, bivalirudin, dabigatran, dabigatran etexilate, ximelagatran, and any salt, derivative or analog thereof. In some embodiments, tetracycline or a derivative, salt, or analog thereof is covalently conjugated to an anticoagulant via a linker. The linker can be an acid labile linker, an ester linker, a hydrazone linker, a sulfonamide-containing linker, an enzymatically cleavable linker, or a polymer based linker. The chemical reactions necessary to carry out the coupling of tetracycline conjugates are standard techniques and are known to one skilled in the art.

The resultant tetracycline-anticoagulant conjugate agent binds, penetrates, disassembles, prevents or disrupts a plaque, and inhibits thrombosis. The plaque-related disease or disorder that can be prevented or treated using the conjugates synthesized via the method of the present invention includes but is not limited to atherosclerosis, atherothrombosis, Alzheimer's disease, Parkinson's disease, osteoarthritis, kidney stone, gall bladder stone, pancreatic stone, or a calcification-related disease.

### B. Method of Preventing or Treating a Plaque-Related Disease or Disorder

In some embodiments, the present invention provides a method of preventing or treating a plaque-related disease comprising administering to a subject an effective amount of a conjugate agent or a pharmaceutical composition thereof, wherein the conjugate agent comprises tetracycline or a derivative, salt, or analog of tetracycline conjugated to an anticoagulant. The subject can be a mammal, preferably a human, more preferably a patient.

The invention encompasses both prophylactic and therapeutic treatment of a plaque-related or plaque-induced disease or disorder. The term “patient” refers to a warm-blooded mammal, preferably a human, who is healthy or who is afflicted with, at risk of being afflicted with, or suspected to be afflicted with, an underlying disease. For example, the composition of the present invention may prevent or disrupt the formation of atherosclerotic plaques, plaque-like complexes, or thromboses. The method may comprise administering to a patient or a test subject or animal a therapeutically-effective amount of a tetracycline conjugate, for example, a tetracycline anticoagulant conjugate described herein. In some embodiments, the tetracycline conjugate is tetracycline conjugated to an anticoagulant molecule. In certain embodiments, the tetracycline conjugates are modified for optimal efficiency. The tetracycline derivative can be selected from 6-demethyltetracycline, bromotetracycline, chlorotetracycline, clomocycline, demeclocycline, doxycycline, lymecycline, meclocycline, methacycline, minocycline, oxytetracycline, rolitetracycline, tetracycline, sancycline, 5a,6-anhydrotetracycline, DDA-tetracycline, dactylocyclinone and 8-methoxychlorotetracycline. The tetracycline derivative also includes the optical isomers of the compounds mentioned above. More preferably, the tetracycline derivative is selected from doxycycline, methacycline, sancycline and minocycline. Even more preferably, the tetracycline derivative is selected from 7-halosancycline, 9-halosancycline and minocycline. Most preferably, the tetracycline derivative is minocycline or any tetracycline derivative or analog or tetracycline conjugated to anti-coagulants (e.g, warfarin, acenocoumarol, clopidogrel).

The term “treat” or its grammatical equivalents as used herein, means providing a therapeutic benefit and/or a prophylactic benefit. A therapeutic benefit refers to the eradication or amelioration of the underlying disorder being treated. Also, a therapeutic benefit is achieved when an improvement is observed in the patient, notwithstanding that the patient may still be afflicted with the underlying disorder. The term “disorder” is used interchangeably with the term “disease” herein. A prophylactic benefit arises when the compositions are administered to a patient at risk of developing a particular disease, or to a patient reporting one or more of the physiological symptoms of a disease, even without a diagnosis.

In some embodiments, the present invention comprises methods of treating, or protecting against, a plaque-related disease including but not limited to atherosclerosis, atherothrombosis, Alzheimer's disease, Parkinson's disease, osteoarthritis, type II diabetes, dementia, atrial amyloidosis, systemic amyloidosis, dialysis-related amyloidosis, hemodialysis-related amyloidosis, fibrinogen α-chain amyloidosis, kidney stone, gall bladder stone, pancreatic stone, or a calcification-related disease in a subject. In a preferred embodiment, the subject is a human suffering from atherothrombosis or atherothrombosis-like processes or at risk of same. In another preferred embodiment, the subject is a human suffering from atherosclerotic diseases or disease-like processes or at risk of same. In another embodiment, the subject is healthy.

In some embodiments, the present invention provides administering to a subject in need thereof a therapeutically effective amount of a tetracycline conjugate that binds, penetrates, disassembles, prevents or disrupts a plaque involved in a plaque-related disease. In one embodiment, the tetracycline conjugate is a tetracycline-anticoagulant conjugate. The anticoagulant can be selected from the group consisting of clopidogrel, warfarin (coumadin), acenocoumarol, enoxaparin, prasugrel, phenprocoumon, brodifacoum, phenindione, heparin, fondaparinux, idraparinux, argatroban, lepirudin, bivalirudin, dabigatran, dabigatran etexilate, ximelagatran, and any salt, derivative or analog thereof. In other embodiments, tetracycline or a derivative or analog thereof can be conjugated to a molecule including but not limited to a cytotoxin, radioisotope, hormone such as a steroid, anti-metabolite, cytokine, growth factor, or chemotherapeutic agent. The tetracycline moiety of the conjugate targets the molecule which is conjugated to tetracycline to the sites of plaques, for example, advanced atherosclerotic plaques, thereby allowing more effective prevention or treatment of a plaque-related disease or disorder.

For embodiments where a prophylactic benefit is desired, a pharmaceutical composition of the invention may be administered to a patient at risk of developing a plaque-related disease, or to a patient reporting one or more of the physiological symptoms of a plaque-related disease, even though a diagnosis of the condition may not have been made. Administration may prevent the plaque from developing, or it may reduce, lessen, shorten and/or otherwise ameliorate the plaque that develops. The pharmaceutical composition may modulate a plaque activity or stability. Wherein, the term “modulate” includes inhibition or alternatively activation of a molecule that affects the plaque.

In some embodiments, the tetracycline-drug conjugate exhibits reduced side effects and improved pharmacokinetic properties as compared to unconjugated tetracycline and the drug when administered individually. In one example, the tetracycline-anticoagulant conjugate exhibits reduced side effects and toxicity as compared to unconjugated tetracycline and the anticoagulant when administered individually. In another example, the tetracycline-anticoagulant conjugate increases half-life of the anticoagulant as compared to administration of the anticoagulant alone. The administration of the tetracycline conjugate can be oral or parenteral injection.

In further embodiments, atherosclerotic plaques described herein are introduced into the system of a subject, including a human or a non-human animal. In one embodiment, the tetracycline conjugates of the present invention are pre-screened for their ability to treat, reduce, or prevent atherosclerotic disease or disease-like processes. In another embodiment, the tetracycline conjugates of the present invention are sequentially or contemporaneously introduced into the system of a live non-human animal or a human in order to protect against various stages of a plaque-related disease.

### Administration of the Composition:

In one aspect, this disclosure provides a method of preventing or treating a plaque-related disease comprising administering to a subject an effective amount of a conjugate or a pharmaceutical composition thereof, wherein the conjugate comprises tetracycline or a derivative, salt, or analog of tetracycline conjugated to an anticoagulant. In some embodiments, the subject is an animal including mammal. In some embodiments, the animal exhibits one or more signs or symptoms of a plaque-related disease. The administration with the tetracycline conjugates of the present invention into the subject prevents, alleviates, or treats one or more signs or pathological symptoms associated with a plaque-related disease including but not limited to atherosclerosis, atherothrombosis, coronary artery disease, Alzheimer's disease, Parkinson's disease, cerebral hemorrhage with amyloidosis, atrial amyloidosis, hemodialysis-related amyloidosis, or amyotrophic lateral sclerosis. The animal used in the present invention may be a mouse, rat, pig, horse, non-human primate, guinea pig, hamster, chicken, frog, cat, dog, sheep, or cow. In one embodiment, the subject is a human, either healthy or with a plaque-related disease.

In the method of the invention, the subject is administered an amount of a tetracycline conjugate, for example, a tetracycline-anticoagulant conjugate in one or several dosages. The dosage depends upon the purity and chemical form of the tetracycline conjugate and the degree of absorption expected. Suitable amounts per dose are typically greater than about 0.1 mg/kg of body weight, preferably in the range of from about 0.1 to 5.0 mg/kg per dose, and most preferably are about 0.1 to 2.0 mg/kg per dose. These dosage ranges are intended to be suggestive and should not necessarily be considered as limiting, since the individual reactions of particular subjects will vary. Adjustment of the dosage ranges in accordance with individual variations is routine among practitioners.

Similarly, no single protocol appears to be desirable for all cases at this time. However, typical protocols will include either a single dose or an initial dose followed by 1-4 additional doses at weekly, biweekly or monthly intervals. In one embodiment, the protocol calls for 3 doses about two weeks apart. Again, these protocols are not intended to be limiting in view of the wide variation permitted in protocol design.

The tetracycline conjugate of the invention may be administered as a single compound, or as a mixture of various anti-plaque agents. Suitable formulations include those appropriate for systemic administration, including preparations for parenteral injection or infusion (intravenous, intraperitoneal, intramuscular, intraarterial, intracardiac, intraosseous infusion, intradermal, intrathecal, intravesical, or subcutaneous), transmucosal or transdermal administration, inhalation, or oral administration. One example of formulating the tetracycline conjugate of the invention for this use is in the form of a solution or as liposomes suitable for injection into a peripheral vein. Suitable methods for preparing liposomes are well-known in the art.

When injected intravenously, the solution or liposome formulation disseminates throughout the vascular system and thus comes into direct contact with the arterial plaques being targeted, where the tetracycline conjugate is selectively absorbed. The presence of the tetracycline conjugate in the plaque may be detected within a few hours after injection and may persist for as long as several days to two weeks.

In one embodiment, the targeting of tetracycline conjugate to atheromatous plaques may be enhanced by binding to the tetracycline conjugate some antibody specific to a component of the plaque. Monoclonal antibodies may be particularly useful due to their extreme specificity. Components of plaque that can serve as antigenic targets include elastic elements, collagen, and lipid constituents.

### EXAMPLES

The following non-limiting examples further illustrate the invention disclosed herein:

### Example 1

### Tetracycline-Clopidogrel Bisulfate Conjugate

In this example, tetracycline or its non-antibiotic (CMT-3) derivative is conjugated to clopidogrel bisulfate, which is an oral antiplatelet agent (thienopyridine class) to inhibit blood clots in coronary artery disease, peripheral vascular disease, and cerebrovascular disease. The conjugate has properties to have high binding affinity to the advanced atherosclerotic plaques and inhibition of blood platelet aggregation. Clopidogrel irreversibly inhibits blood platelet activation by blocking adenosine 5′-diphosphate (ADP) receptor of blood platelets (Chopra et al.).

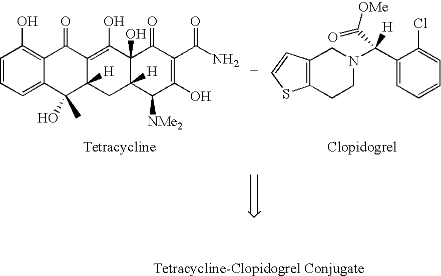

Since tetracyclines, for example doxycycline, are widely being used to treat bacterial infections, antibacterial resistance is always of concern for tetracycline-containing agents. Thus the preferred scaffold to use for synthesizing anticoagulant conjugates is a tetracyclic scaffold that has no intrinsic antibacterial activity. Notable examples are 4-dedimethylaminosancycline (CMT-3) or 4-dedimethylaminodoxycycline which is semi-synthetically attained from sancycline or doxycycline, respectively. CMT-3 has novel anti-inflammatory, anti-fungal, anti cancer properties (Yu Liu et al, 2002; Colub L M et al., 1986).

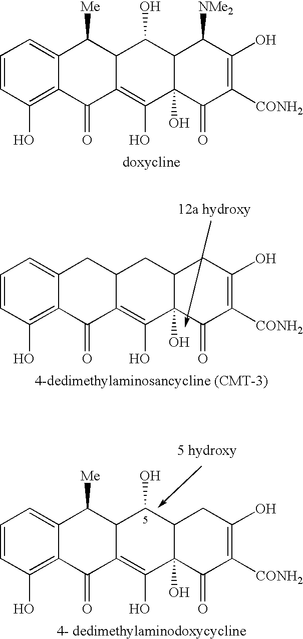

### Examples of Tetracycline and Non-Antibody Scaffold of Tetracycline

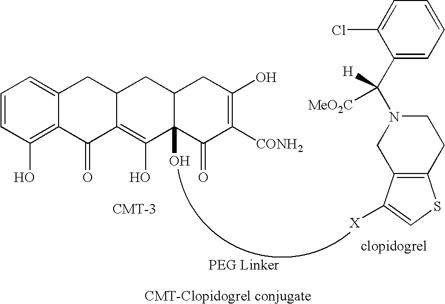

### Example 2

### Tetracycline-Acenocoumorol Conjugate

In this example, tetracycline is conjugated to acenocymorol, which is a member of coumarin family referred to as vitamin K antagonists. These drugs indirectly prevents blood coagulation by inhibiting vitamin K epoxide reductase, an enzyme that recycles oxidated vitamin K to its reduced form after it has participated in the carboxylation of several blood coagulation proteins, mainly prothrombin and factor VII (Ansell et al.).

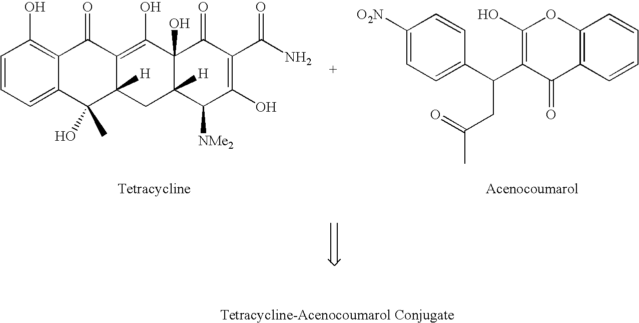

### Example 3

### Tetracycline-Dabigatran Etexilate Conjugate

In this example, tetracycline or CMT-3 is conjugated to dabigatran, which is an anticoagulant that directly inhibits activity of a critical blood coagulation enzyme, thrombin. Currently, it is orally administered as the prodrug dabigatran etexilate for thrombosis related indications (Eriksson et al.).

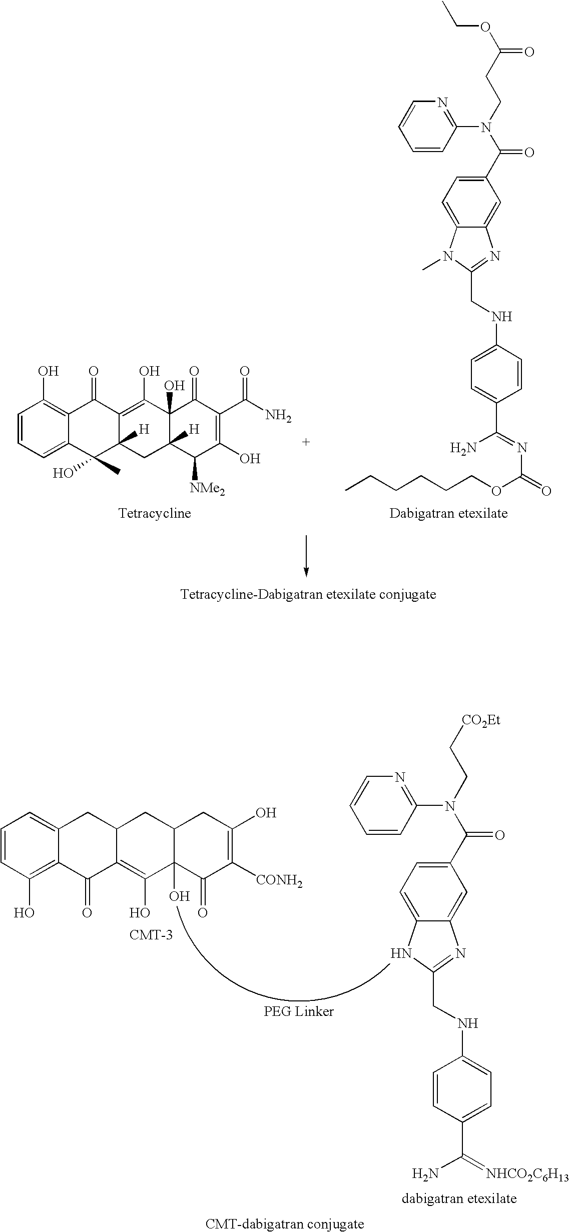

### Example 4

### Heparin-Tetracycline Conjugate

In this example, tetracycline is conjugated to heparin, which is an indirect thrombin inhibitor which complexes with antithrombin (ATIII), a physiological inhibitor of blood coagulation factors. Heparin is a cofactor and its binding to ATIII leads to rapid inactivation of a number of coagulation factors such as thrombin, factor Xa, and to a lesser extent, factors XIIa, XIa, and IXa (Hirsh et al.).

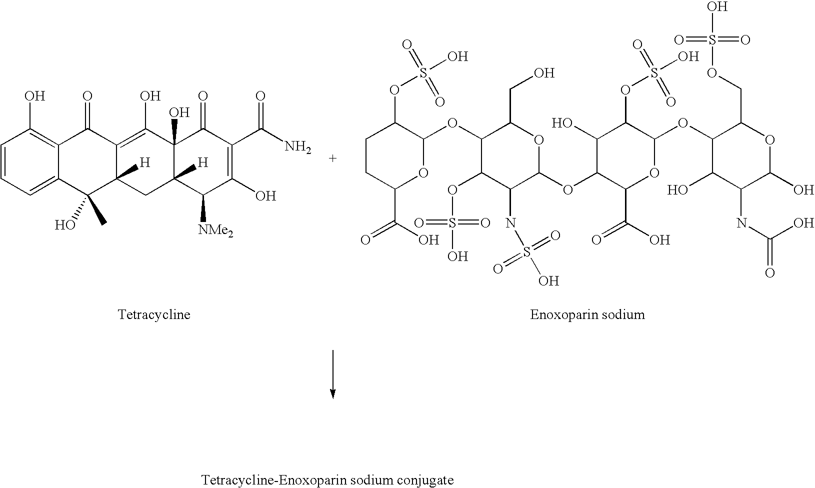

### Example 5

### Tetracycline-Prasugrel Conjugates

In this example, tetracycline is conjugated to prasugrel, which is a member of blood platelet ADP receptor inhibitor like clopidogrel and ticlopidine. These drugs inhibit aggregation of blood platelet by irreversibly binding to P2Y12 receptors (Wiviott et al.). Parasugrel is currently under clinical development for treating percutaneous coronary intervention.

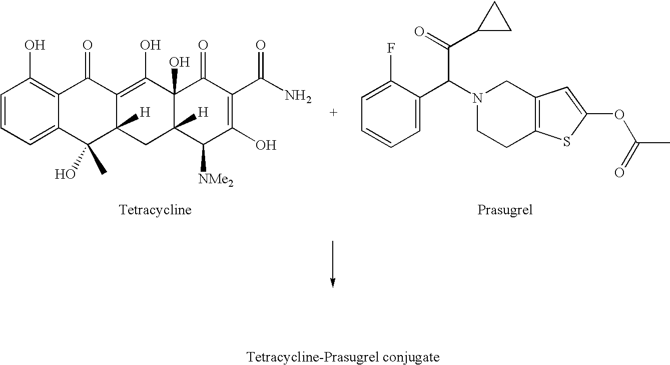

### Example 6

### Inhibition of Thrombosis of Plaque Complexes by Tetracycline-Clopidogrel Conjugate

This example illustrates the inhibition of thrombosis of aggregates/plaque complexes by tetracycline-clopidogrel conjugate. The tetracycline-clopidogrel conjugate is used to examine its effect on in vitro and in vivo thrombosis studies.

### Testing of Conjugates in Biochemical and Cell Culture Plaque Assays

The conjugates will be screened in the atherosclerosis biochemical plaque assays. The conjugates will be tested for dual function. Following tests will be performed and molecules will be identified for further cell culture testing. Experiments are performed with the tetracycline-clopidogrel conjugate to examine its effect on thrombosis in vitro. The calcium phosphate (CP) based plaque like complexes are used in the thrombosis assay as described in Section 2.4.1 of U.S. patent application Ser. No. 12/286,368, which is herein incorporated by reference in its entirety. The CP, CP-Cholesterol (Chl), and CP-Lipids based plaque complexes (100 μl), generated after four cycles of cyclic plaque assembly, are treated with 2 mM tetracycline-clopidogrel conjugate at 37° C. for 30 min followed by centrifugation. After discarding the supernatant, the CP-tetracycline-clopidogrel conjugate, CP-Chl-tetracycline-clopidogrel conjugate and CP-Lipids-tetracycline-clopidogrel conjugate complexes are used for thrombosis using prothrombin (3 μg) and fibrinogen (4 μg) or 2.5% human plasma. For control experiment, the CP without the tetracycline-clopidogrel conjugate treatment is used for thrombosis. The results show that the CP based plaque complexes treated with the tetracycline-clopidogrel conjugate do not form thrombus and, as expected, thrombosis is observed in the control experiment without the tetracycline-clopidogrel conjugate. These experiments suggest that the tetracycline-clopidogrel conjugate inhibits thrombosis of the plaque like complexes. The tetracycline-clopidogrel conjugate may inhibit the activity of the thrombin, a member of serine protease, thereby inhibiting the coagulation of plaque complexes.
a. Binding of conjugates in biochemical plaque assays (early stage and late stage plaque complexes)
b. Testing for binding with human Atherosclerotic plaques (Human Atherosclerotic plaques to be purchased from Curaline, Inc)
c. Activation of conjugates and testing the active metabolite in in vitro plaque thrombosis assays
d. ADP receptor binding assay
e. Testing of active metabolites/conjugates for anti-platelet aggregation function using human blood samples

### Characterization of Conjugates in Cell Culture Assays

The conjugates, Tetracycline-Cloipidogrel or Tetracycline-dabigatran or Tetracycline-acenocumorol will be further evaluated using the following tests. The calcium phosphate (CP) based plaque like complexes are used in the cell culture assay as described in U.S. patent application Ser. No. 12/286,368, which is herein incorporated by reference in its entirety. These assays will further narrow down the positive candidate and will be an important Go/No go decision point for each candidate.
a. Cytotoxic assays for 4-6 conjugates with Human Artery endothelial cells, HUVEC and/or Monocytes
b. Cytokine profiles
c. Cell-Plaque thrombosis assay
d. Apoptosis (cell mortality)

### Example 7

### Inhibition of Plaque Development in Induced Mouse Models of Atherosclerosis

Inducing atherosclerotic plaque development in mice is disclosed in details in U.S. patent application Ser. No. 12/286,368, which is herein incorporated by reference in its entirety. These mouse models are used for testing tetracycline-acenocoumorol conjugate to determine their efficacy both on reducing progression of plaque development and symptoms of chronic inflammation. Mice purchased from vendors are used for in vivo atherosclerotic plaque development by multiple doses of plaque complexes by intra-peritoneal and intravenous routes as described U.S. patent application Ser. No. 12/286,368. After confirming plaque growth in the heart tissue and chronic inflammatory symptoms, the mice are treated with or without tetracycline-acenocoumorol conjugate of the present invention. Ovastatin, an established anti-atherosclerotic drug (Schwartz et al, 2001) is used for control purposes in these studies and administered by oral route.

Ovastatin, tetracycline, clopidogrel or any anti-coagulant individually and tetracycline-clopidogrel (0.1-5 mg/kg per day) are orally administrated to the symptomatic and asymptomatic atherosclerotic mice models for 8 to 12 weeks. Following administration of the drugs, mice with asymptomatic plaque development are euthanized and blood collected via cardiac puncture. The blood samples will be used test clotting or blood coagulation test to analyse anti-coagulation efficacy of drugs used. In addition, heart and arteries are isolated from these mice and used for biochemical and histochemical analysis to confirm the efficacy of these drugs on atherosclerotic plaque development. As compared to mice receiving clopidogrel alone, the mice that received equivalent amount of the tetracycline-clopidogrel conjugate show reduced systemic bleeding and less severe ischemic events.

Mice showing morphological plaque induced chronic inflammation are monitored after treatment with the tetracycline-acenocoumorol or tetracycline-clopidogrel conjugate for reduction in the symptoms such as healing of skin redness, reduced scratching, re-growing of hairs and improved activity compared to control mice without the conjugate treatment. The mice treated with ovastatin or tetracycline-acenocoumorol conjugate show reduced symptoms of plaque inflammation compared to the control animal without the treatment. In addition, histochemical analysis of the heart tissues isolated from asymptomatic mice model show reduced plaque accumulation after treating with both ovastatin and the tetracycline-clopidogrel or tetracycline-acenocumorol conjugate.

The atherosclerotic mouse models developed in this study mimic advanced atherosclerotic plaques development and suitable for testing efficacy of anti-atherosclerotic drug candidates on both plaque regression, induced thrombosis and chronic plaque inflammation. Drugs showing efficacy on blood coagulation, anti-platelet aggregation, plaque regression and plaque inflammation are expected to be more effective to treat patients with atherosclerosis.

For analyzing the pharmacokinetic (PK) parameters of the tetracycline-clopidogrel or tetracycline-acenocumorol conjugate, induced mice weighing 20-40 g are acclimatized for 2-3 days with a 12-hour light-dark cycle at a temperature of 20-23° C. with free access to standard rodent chow and water ad libitum. The tetracycline-clopidogrel or tetracycline-acenocoumorol conjugate is administered by oral (op), intravenous (iv), intra-peritoneal (ip) and/or subcutaneous (sc) routes. Following administration of the tetracycline-clopidogrel or tetracycline-acenocoumorol conjugate, blood samples are collected at various time points and used for biochemical analysis to determine their efficacy towards thrombosis. Each mouse is euthanized by carbon-dioxide inhalation and blood is collected by cardiac puncture and/or other tissues are removed for analyzing binding of tetracycline-clopidogrel conjugate. In addition, histological studies are carried out to determine plaque regression in the drug administrated animals. Based on the PK results, the tetracycline-acenocoumorol conjugate exhibits improved PK characteristics, for example, increased half life, as compared to the PK profile of unconjugated acenocoumorol administered alone.

The biochemical studies in vitro and in vivo suggest that preventing the coagulation of plaque complexes can reduce the risks and mortalities of patients who are diagnosed with plaque-related diseases, such as advanced atherosclerosis and atherothrombosis. As demonstrated in Example 6, the tetracycline conjugate of the present invention can inhibit thrombosis of calcium phosphate (CP) based plaques. The CP based pathologies are associated with numerous human diseases, particularly, 82% of the patients with advanced atherosclerosis have higher accumulation of the CP with co-localized lipids and cholesterol crystals. In addition, the coronary calcification associated with aortic valve calcification is the third leading cause of the heart diseases in adults (Garg V et al 2005) and approximately 40-50% of mortalities of patients with chronic kidney disease are due to atherosclerosis, valvular calcification, myocardial infarction etc (Campean V et al, 2005). Treating patients diagnosed with these aforementioned diseases with the tetracycline conjugates of the present invention would reduce the risks associated with these diseases and result in higher efficacy of the treatment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

1. Wodlinger A M, Pieper J A. The role of clopidogrel in the management of acute coronary syndromes. Clin Ther. 2003, (8):2155-81.
2. Chu M W, Wilson S R, Novick R J, Stitt L W, Quantz M A. Does clopidogrel increase blood loss following coronary artery bypass surgery? Ann Thorac Surg. 2004, 78(5):1536-41.
3. Pickard A S, Becker R C, Schumock G T, Frye C B. Clopidogrel-associated bleeding and related complications in patients undergoing coronary artery bypass grafting. Pharmacotherapy. 2008, 28(3):376-92.
4. DeEugenio D, Kolman L, DeCaro M, Andrel J, Chervoneva I, Duong P, Lam L, McGowan C, Lee G, DeCaro M, Ruggiero N, Singhal S, Greenspon A. Risk of major bleeding with concomitant dual antiplatelet therapy after percutaneous coronary intervention in patients receiving long-term warfarin therapy. Pharmacotherapy. 2007, 27(5):691-6.
5. Corti, R., Hutter R., Badimon, J. J., Fuster, V. Evolving concepts in the triad of atherosclerosis, inflammation and thrombosis. J Thromb Thrombolysis. 17. 35-44 (2005).
6. Doherty, T. M., Fitzpatrick, L. A., Inoue, D., et al, Molecular, endocrine and genetic mechanisms of arterial calcification. Endocrine reviews. 25, 629-672 (2003).
7. Mykkanen, L., Kuusisto, J., Haffner, S. M., Laakso, M., Austin, M. A. LDL size and risk of coronary heart disease in elderly men and women. Arterioscler Thromb Vasc Biol. 19, 2742-2748 (1999).
8. Carmena, R., Duriez, P., Fruchart, J. C. Atherogenic lipoprotein particles in atherosclerosis. Circulation. 109, (suppl III-2-III-7) (2004).
9. Hirona, T., Ito, Y., Koba, S., Toyoda, M., Ikejiri, A., Saegusa, H., Yamazaki, J., Yoshino, G. Clinical significance of small dense low-density lipoprotein cholesterol levels determined by the simple precipitation method. Arterioscler Thromb Vasc Biol. 24, 558-563 (2004).
10. Festa, A., Agostino, R. D., Mykkanen, L., Tracy, R., Howard, B. V., Haffner, S. V. Low-density lipoprotein particle size is inversely related to plasminogen inhibitor-1 levels. Arterioscler Thromb Vasc Biol. 19, 605-610 (1998).
11. Peng, S., Guo, W., Morrisett, J. D., Johnstone, M. T., Hamilton, J. A. Quantification of cholesteryl esters in human and rabbit atherosclerotic plaques by magic-angle spinning. Arterioscler Thromb Vasc Biol. 12. 2682-8 (2000).
12. Sarig, S., Utian, W. H., Sheean, L. A., Gorodeski, G. I. Distribution of unesterified cholesterol-containing particles in human atherosclerotic lesions. Am. J. Pathol. 146, 139-147 (1994a).
13. Small, D. M., Progression, and regression of atherosclerotic lesions: insights from lipid physical biochemistry. Arteriosclerosis, 8, 103-129 (1988).
14. Guyton, J. R., Klemp, K. F. Development of the lipid-rich core in human atherosclerosis. Arterioscler Thromb Vasc Biol. 16, 4-11 (1996).
15. Sarig, S., Weiss, T. A., Katz, I, Kahana, F., Azoury, R., Okon, E., Kruth, H. S. Detection of cholesterol associated with calcium mineral using confocal fluorescence microscopy. Lab Invest, 71, 782-787 (1994b).
16. Kruth, H. S., Filipin-positive, oil red O-negative particles in atherosclerotic lesions induced by cholesterol feeding. Lab invest. 50, 87-93 (1984).
17. Hirsch, D., Azoury, R., Sarig, S., Kruth, H. S. Colocalization of cholesterol and hydroxyapatite in human atherosclerotic lesions. Calcif Tissue Int. 52, 94-98 (1993).
18. Guo, W., Morrisett, J. D., DeBakey, M. E., Lawrie, G. M., Hamilton, J. A. Quantitation in situ of crystalline cholesterol and calcium phosphate hydroxyapatite in human atherosclerotic plaques by solid state magic angle spinning NMR. Arterioscler Thromb Vasc Biol. 20, 1630-1636 (2000).
19. Brancaccio, D., Cozzolino, M. The mechanism of calcium deposition in soft tissues. Contrib Nephrol. 149, 279-86 (2005).
20. Young, E. W., Albert, J., M., Satayathum, S., Goodkin, D. A., Pisoni, R. L., Akiba, T., Kurokawa, K., Bommer, J., Piera, L., Poit, F. K. Predictors and consequences of altered mineral metabolism: the dialysis outcomes and practice patterns study. Kidney Int. 67, 1179-87 (2005).
21. Carson, D. A. An infectious origin of extraskeletal calcification. Proc Natl Acad. Sci. 95, 7846-7847 (1998).
22. Bini, A., Mann, K. G., Kudryk, B. J., Schoen, F. J. Noncollagenous bone matrix proteins, calcification, and thrombosis in carotid artery atherosclerosis. Arterioscler Thromb Vasc Biol. 19. 1852-61 (1999).
23. Garg, V., Muth, A. N., Ransom, J. F., Schluterman, M. K., Barnes, R., King, I. N., Grossfeld, P. D., Srivastava, D. Mutations in NOTCH1 cause aortic valve disease, Nature. 437, 270-273 (2005).
24. Campean, V., Neureiter, D., Varga, I., Runk, F., Reiman, A., Garlichs, C., Achenbach, S., Normast-Daniel, B., Amann, K. Atherosclerosis and vascular calcification in chronic renal failure. Kidney Blood Press Res. 28. 280-9 (2005).
25. Cullen, P., Rauterberg, J., Lorkowski, S. The pathogenesis of atherosclerosis. Handb Exp Pharmacol. 170. 3-70 (2005).
26. Xiao, Q., Danton, M. J., Witte, D. P., Kowala, M. C., Valentine, M. T., Degen, J. L. Fibrinogen deficiency is compatible with the development of atherosclerosis in mice. J Clin Invest. 101. 1184-94 (1998).
27. Savi, P; et al. Proceedings of the National Academy of Sciences of the USA. 2006, 103 (29): 11069-11074. Chopra V, Marmur J D, Cavusoglu E. The role of clopidogrel in the management of patients with ischemic heart disease. Cardiovasc Drugs Ther. 2003, 17(5-6):467-77.
28. Yu L., Maria E R., His-Ming L., Sanford M., George T., Carol L., Michael K L., Golub L. M. A chemically modified tetracycline (CMT-3) is a new antifungal agent. Antimicrob Agents Chemother. 46, 1447-1454 (2002).
29. Collub T., McNamara T F., Angelo D., Greenwald R A., Ramamurthy N S. A non-antibacterial chemically modified tetracycline inhibits mammalian collagenase activity. J Dent Res. 66, 1310-1314 (1987).
30. Ansell J, Hirsh J, Poller L, Bussey H, Jacobson A, Hylek E (2004). “The pharmacology and management of the vitamin K antagonists: the Seventh ACCP Conference on Antithrombotic and Thrombolytic Therapy”. Chest 126 (3 Suppl): 204S-233S.
31. Eriksson B I, Dahl O E, Ahnfelt L, et al (September 2004). “Dose escalating safety study of a new oral direct thrombin inhibitor, dabigatran etexilate, in patients undergoing total hip replacement: BISTRO I”. J. Thromb. Haemost. 2 (9): 1573-80
32. Hirsh, J, Anand, S S, Halperin, J L, Fuster, V. Guide to anticoagulant therapy: heparin: a statement for healthcare professionals from the American Heart Association. Circulation 2001; 103:2994.
33. Wiviott S D, Braunwald E, McCabe C H, et al. (2007). “Prasugrel versus clopidogrel in patients with acute coronary syndromes”. N Engl J Med 357 (20): 2001-15.
34. Garnett M C. Targeted drug conjugates: principles and progress. Adv Drug Deliv Rev 2001; 53(2): 171-216.
35. Eaton M. Immunoconjugates: current status and future potential. J Drug Target 2002; 10(7): 525-527.
36. Kratz F, Beyer U, Schutte M T. Drug-polymer conjugates containing acid-cleavable bonds. Crit Rev Ther Drug Carrier Syst 1999; 16(3): 245-288.
37. Toth I. A novel chemical approach to drug delivery: lipid amino acid conjugates. J Drug Target 1994; 2(3): 217-239.
38. Lavie E, Hirschberg D L, Schreiber G, Thor K, Hill L, Hellstrom I, et al. Monoclonal antibody L6-daunomycin conjugates constructed to release free drug at the lower pH of tumor tissue. Cancer Immunol Immunother 1991; 33(4): 223-230.
39. Shen W C, Ryser H J. cis-Aconityl spacer between daunomycin and macromolecular carriers: a model of pH-sensitive linkage releasing drug from a lysosomotropic conjugate. Biochem Biophys Res Commun 1981; 102(3): 1048-1054.
40. Hudecz F, Ross H, Price M R, Baldwin R W. Immunoconjugate design: a predictive approach for coupling of daunomycin to monoclonal antibodies. Bioconjug Chem 1990; 1(3): 197-204.
41. Blättler W A, Kuenzi B S, Lambert J M, Senter P D. New Heterobifunctional Protein Cross-Linking Reagent That Forms An Acid-Labile Link. Biochemistry 1985; 24(6): 1517-1524.
42. Srinivasachar K, Neville D M, Jr. New protein cross-linking reagents that are cleaved by mild acid. Biochemistry 1989; 28(6): 2501-2509.
43. Warnecke A, Kratz F. Maleimide-oligo(ethylene glycol) Derivatives of Camptothecin as Albumin-Binding Prodrugs: Synthesis and Antitumor Efficacy. Bioconjug Chem 2003; 14(2): 377-387.
44. Dubowchik G M, Walker M A. Receptor-mediated and enzyme dependent targeting of cytotoxic anticancer drugs. Pharmacol Ther 1999; 83(2): 67-123.
45. Chari R V. Targeted delivery of chemotherapeutics: tumor-activated prodrug therapy. Adv Drug Deliv Rev 1998; 31(1-2): 89-104.
46. Garnett M C. Targeted drug conjugates: principles and progress. Adv Drug Deliv Rev 2001; 53(2): 171-216.
47. Kratz F, Beyer U, Schutte M T. Drug-polymer conjugates containing acid-cleavable bonds. Crit Rev Ther Drug Carrier Syst 1999; 16(3): 245-288.
48. Greenwald R B, Choe Y H, McGuire J, Conover C D. Effective drug delivery by PEGylated drug conjugates. Adv Drug Deliv Rev 2003; 55(2): 217-250.
49. Guo P, Ma J, Li S, Guo Z, Adams A L, Gallo J M. Targeted delivery of a peripheral enzodiazepine receptor ligand-gemcitabine conjugate to brain tumors in a xenograft model. Cancer chemother Pharmacol 2001; 48(2): 169-176.
50. Guo Z, Gallo J M. Selective Protection of 2′,2′-Difluorodeoxycytidine (Gemcitabine). J Org Chem 1999; 64(22):8319-8322.
51. Sum, F.-W.; Wong, V.; Han, S.; Largis, E.; Mulvey, R.; Tillett, J. Cyclic amine sulfonamides as linkers in the design and synthesis of novel Human b3 Adrenergic Receptor Agonists. Bioorg. Med. Chem. Lett. 2003, 13, 2191-2194
52. Comparison of kinetic properties of native and recombinant human cathepsin D. Adv Exp Med Biol 1991; 306: 343-347.
53. Scarborough P E, Guruprasad K, Topham C, Richo G R, Conner G E, Blundell T L, et al. Exploration of subsite binding specificity of human cathepsin D through kinetics and rule-based molecular modeling. Protein Sci 1993; 2(2): 264-276.
54. Papot S, Tranoy I, Tillequin F, Florent J C, Gesson J P. Design of selectively activated anticancer prodrugs: elimination and cyclization strategies. Curr Med Chem Anti-Canc Agents 2002; 2(2): 155-185.
55. Saito G, Swanson J A, Lee K D. Drug delivery strategy utilizing conjugation via reversible disulfide linkages: role and site of cellular reducing activities. Adv Drug Deliv Rev 2003; 55(2): 199-215.
56. Sawant R R, Sawant R M, Torchilin V P. Mixed PEG-PE/vitamin E tumor-targeted immunomicelles as carriers for poorly soluble anti-cancer drugs: improved drug solubilization and enhanced in vitro cytotoxicity. Eur J Pharm Biopharm. 2008 September; 70(1):51-7.
57. Schwartz, G. G., Olsson, A. G., Ezekowitz, M. D., Ganz, P., Oliver, M. F., Waters, D., Zeiher, A., Chaitman, P. R., Leslie, S., Stern, T. (2001) Effects of Atorvastatin on Early Recurrent Ischemic Events in Acute Coronary Syndromes. JAMA, 285:1711-1718

What is claimed is:

1. A composition for preventing or treating a plaque-related disease, the composition comprising tetracycline or a derivative, salt, or analog of tetracycline conjugated to an anticoagulant.

2. The composition of claim 1, wherein the tetracycline salt is tetracycline chloride.

3. The composition of claim 1, wherein the anticoagulant is selected from the group consisting of clopidogrel, warfarin (coumadin), acenocoumarol, elinogrel, enoxaparin, prasugrel, phenprocoumon, brodifacoum, phenindione, heparin, fondaparinux, idraparinux, argatroban, lepirudin, bivalirudin, dabigatran, dabigatran etexilate, ximelagatran, and any salt, derivative or analog thereof.

4. The composition of claim 1, wherein the tetracycline or a derivative, salt, or analog of tetracycline is a non-antibiotic form of tetracycline.

5. The composition of claim 1, wherein the tetracycline or a derivative, salt, or analog of tetracycline is covalently conjugated to an anticoagulant via a linker.

6. (canceled)

7. The composition of claim 1, wherein the tetracycline-anticoagulant conjugate binds, penetrates, disassembles, prevents or disrupts a plaque.

8. (canceled)

9. (canceled)

10. (canceled)

11. The composition of claim 1, wherein the plaque-related disease is selected from the group consisting of atherosclerosis, atherothrombosis, Alzheimer's disease, kidney stone, gall bladder stone, pancreatic stone, and a calcification-related disease.

12. The composition of claim 1 further comprising a pharmaceutically acceptable excipient or carrier.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method of preventing or treating a plaque-related disease or disorder comprising administering an effective amount of tetracycline, or a derivative, salt, or analog thereof, to a subject afflicted with, at risk for, or suspected of being afflicted with, a plaque-related disease or disorder.

31. The method of claim 30, wherein said tetracycline or a derivative, salt, or analog thereof, is conjugated to an anticoagulant.

32. The method of claim 30, wherein the tetracycline salt is tetracycline chloride.

33. The method of claim 31, wherein the anticoagulant is selected from the group consisting of clopidogrel, warfarin (coumadin), acenocoumarol, elinogrel, enoxaparin, prasugrel, phenprocoumon, brodifacoum, phenindione, heparin, fondaparinux, idraparinux, argatroban, lepirudin, bivalirudin, dabigatran, dabigatran etexilate, ximelagatran, and any salt, derivative or analog thereof.

34. The method of claim 30, wherein the tetracycline or a derivative, salt, or analog thereof is conjugated to an anticoagulant via a linker.

35. (canceled)

36. (canceled)

37. The method of claim 31, wherein the tetracycline-anticoagulant conjugate binds, penetrates, disassembles, prevents or disrupts a plaque.

38. The method of claim 31, wherein the tetracycline-anticoagulant conjugate inhibits thrombosis.

39. The method of claim 30, wherein the plaque-related disease or disorder is atherosclerosis.

40. The method of claim 30, wherein the plaque-related disease or disorder is atherothrombosis.

41. The method of claim 30, wherein the plaque-related disease or disorder is selected from the group consisting of atherosclerosis, atherothrombosis, Alzheimer's disease, kidney stone, gall bladder stone, pancreatic stone, and a calcification-related disease.

42. The method of claim 31, wherein the tetracycline-anticoagulant conjugate targets the anticoagulant to sites of plaques.

43. The method of claim 31, wherein the tetracycline-anticoagulant conjugate exhibits reduced side effects, reduced toxicity or reduced bleeding as compared to unconjugated tetracycline and the anticoagulant when administered individually.

44. The method of claim 31, wherein the tetracycline-anticoagulant conjugate improves pharmacokinetic profile of the anticoagulant as compared to administration of the anticoagulant alone.

45. The method of claim 31, wherein the tetracycline-anticoagulant conjugate increases half-life of the anticoagulant as compared to administration of the anticoagulant alone.

46. (canceled)

47. The method of claim 30, wherein the subject is a human.

48. The method of claim 30, wherein the administration of the conjugate is oral or parenteral injection.

49. The method of claim 30, wherein the tetracycline or a derivative, salt, or analog of tetracycline is a non-antibiotic form of tetracycline.
